(19) United States
(12) Patent Application Publication
(10) Pub. No.: US 2004/0023274 A1
(43) Pub. Date: Feb. 5, 2004

(54) Method for the quantification of carbohydrates

(76) Inventor: Yasuro Shinohara, Benten (JP)

Correspondence Address: AMERSHAM BIOSCIENCES, PATENT DEPARTMENT, 800 CENTENNIAL AVENUE, PISCATAWAY, NJ, 08855, US

(21) Appl. No.: 10/416,438

(22) PCT Filed: Nov. 7, 2001
(86) PCT No.: PCT/EP01/12853

(30) Foreign Application Priority Data
Nov. 9, 2000 (SE) 0004094-9

(51) Int. Cl. C12Q 1/68; G01N 33/00

(52) U.S. Cl. 435/6; 436/94

(57) ABSTRACT

The present invention relates to a method for quantification of carbohydrates in two or more starting samples. The method comprises the steps of: (i) providing a combined sample containing for each carbohydrate a mixture of mass tagged form(s) derived from the carbohydrate, wherein each of said one or more mass tagged forms in the mixture: comprises a mass tag unique for the starting sample from which its carbohydrate part is derived, and is present in the combined sample in an amount that relates to the amount of the carbohydrate in the starting sample from which its carbohydrate part is derived; (ii) subjecting each mixture to mass spectrometry to obtain a mass spectrum; (iii) quantifying the amount of a carbohydrate to be quantified in one original sample relative to the amount(s) of the same carbohydrate in one or more of the other original samples. The invention also relates to a kit of reagents useful in the method above.

### TECHNICAL FIELD

The present invention relates to a method for the quantitative determination of one or more carbohydrates (A, B, C etc) in two or more samples (starting samples I, II, III etc).

The carbohydrates concerned typically contain one, two, three or more monosaccharide units.

### BACKGROUND

During the last decades it has become utterly clear that the glycosylation patterns of individual biomolecules often have a tremendous impact on their biological activity. The pattern is reflected on the biomolecules as such, and in free carbohydrates re15 leased from cells and from glycoproteins, glycolipids etc. Qualitative and quantitative variations have been used to diagnose diseases, to monitor metabolic events etc. The diagnostic use has included pure diagnoses in order to check for a certain disease, monitoring of therapy, prognostication of a disease etc.

This has led to the development of methods for determining qualitative and quantitative variations in glycosylation patterns of individual glycosylated biomolecules.

One route has been to identify and quantify differentially glycosylated forms of single parent proteins. This route has utilised separation principles based on chromatography and/or adsorption, such as high-pressure liquid chromatography, ion exchange chromatography/adsorption and affinity chromatography/adsorption, and isoelectric focusing etc for isolating the parent protein. The methods have often been combined with the use of analytically detectable reagents discriminating between different carbohydrate structures. Lectins, sugar specific antibodies etc have been used as reagents.

A second route has been to isolate a desired parent glycoprotein, release and collect the carbohydrate part, and subsequently analyse the released carbohydrate by mass spectrometry, electrophoresis, chromatography etc.

In both alternative separation principles, such as chromatography, electrophoresis and the like has been carried out in one, two and more dimensions.

Other biomolecules such as glycosphingolipids, proteoglycans etc has been analysed in analogous manners.

Tagging of carbohydrate mixtures with chromophores and/or fluorophores for chromatographic or electrophoretic profiling has been described (Hase, Methods of Mol. Biol. 14 (1993) 69-80; Bigge et al., Anal. Biochem 230 (2) (1995) 229-238; Honda et al., Anal. Biochem 180 (2) (1989) 351-357; Shinohara et al., Anal. Chem. 68(15) (1996) 2573-2579); Hu, J. Chromatog. A 705(1) (1995) 89-103; Starr et al., J. Chromatog. A 720 (1-2) (1996) 295-321)

### SUMMARY OF THE PRESENT INVENTION

Despite all recent efforts there is still a need of simplified procedures for quantification cation of individual carbohydrates or groups of carbohydrates in carbohydrate mixtures.

One object of the invention is to provide improvements in regard to one or more of sensitivity, reproducibility, resolution, protocol simplification etc when quantifying carbohydrates in carbohydrate mixtures.

Another object of the invention is to provide improved qualitative and/or quantitative measurements of co- and/or post-translation carbohydrate modifications of individual parent proteins or groups of proteins.

A further object of the invention is an improved method for relating a change in the co- and/or post-translation carbohydrate modification of one or more parent proteins or groups of parent proteins to one or more differences

(a) between samples obtained from an organism that has been subjected to a differential external stimulus,

(b) between samples obtained from cells and organisms having differentially mutated genes,

(c) between samples obtained from an healthy versus a diseased individual or versus an individual to be tested for a disease (e.g. in diagnosis),

(d) between samples obtained for one and the same individual at different occasions (e.g. for monitoring the development or curing of a disease in an individual),

(e) etc.

The objects above, which relate to co- and/or post-translation modification of proteins, also encompass glycosylation of other biomolecules, such as lipids and nonconjugated carbohydrates.

The objects of the invention are achieved as described in the appended claims.

### BRIEF DESCRIPTION OF THE DRAWINGS

FIG. 1 shows the correlation between molar ratio (BXH-G4/BCH-G4) and signal strength ratio (N=2 or 3). Open circles correspond to mean value while bars indicate individual data.

FIG. 2 shows an MS spectrum obtained showing differential display of oligosaccharides using two tagging reagents.

Definitions

The term “individuals” as mentioned above comprises living organisms, in particular single cells and multi-cellular organisms, including animals, such as avians, mammals, amphibians, reptiles, fishes etc and include humans and beetles. The cells may originate from a vertebrate, such as a mammal, or an invertebrate (for instance cultured insect cells), or a microbe (e.g. cultured fungi, bacterial, yeast etc). Included are also plant cells and other kinds of living cells that may or may not be cultured.

### DETAILED DESCRIPTION OF THE INVENTION

The present inventor has now discovered that these objectives at least partially can be met by a method as defined under the heading “Technical field” by properly combining mass tagging with mass spectrometry. A first aspect of the invention thus is an improved variant of these methods and thus is a method for the quantitative determination of one or more carbohydrates (A, B, C etc) in two or more samples (starting samples I, II, III etc). The main characterising feature of this aspect is that the method shall comprise the steps of:

(i) providing a combined sample containing for each carbohydrate a mixture of one or more mass tagged forms derived from the carbohydrate, each of said one or more mass tagged forms in the mixture

comprising a mass tag that is unique for the starting sample from which its carbohydrate part is derived, and

being present in the combined sample in an amount that relates to the amount of the carbohydrate in the staring sample from which its carbohydrate part is derived;

(ii) subjecting each mixture of mass tagged forms in the combined sample to mass spectrometry to obtain a mass spectrum;

(iii) quantifying from signals of the mass tagged forms in the mass spectrum the amount of a carbohydrate to be quantified in one starting sample relative to the amount(s) of the same carbohydrate in one or more of the other starting samples.

The expression “to obtain a mass spectrum” in step (ii) contemplates both that one single mass spectrum is obtained and that separate mass spectra are obtained for each mixture.

In a preferred embodiment step (i) comprises the steps of:

(a) providing two or more staring samples (samples I, II, III etc);

(b) treating each of the starting samples with a sample unique mass tagging reagent (Reagents I, II, III etc) that is capable of transforming each of said carbohydrates to a mass tagged carbohydrate (A-I, B-I, C-I etc in sample I, A-II, B-II, CIII etc in sample II etc);

(c) combining the mass tagged forms obtained in step (b) to a combined sample containing one mixture of mass tagged forms for each of the carbohydrates to be quantified.

By the expressions “sample unique mass tagging reagent” and “a mass tag that is unique for the staring sample from which the mass tagged form derives” are contemplated the carbohydrates in one sample is tagged with a tag that differs in mass with respect to the tags introduced in any of the other samples.

Providing Starting Samples (step (i.a))

The starting samples contain or are suspected to contain one or more carbohydrates to be quantified. One or more of the samples may be a reference or control sample containing a standard/reference amount of one or more of the carbohydrates to be quantified. At least one of the starting samples contains an unknown amount of a carbohydrate to be quantified

The total number of staring samples is at least two and may for instance be up to 5 or up to 10.

The starting samples are in the preferred cases of biological origin. They may be derived from biological fluids, such as cell lysates or cell homogenates, tissue homogenates, fermentation supernatants, body fluids etc. The most important body fluids are blood-derived such as whole blood, serum and plasma, and lachrymal fluid, semen, cerebrospinal fluid (CSF), saliva, urine etc. Samples of biological origin include also any other liquid samples containing bioorganic molecules selected among proteins, carbohydrates, lipids, hormones etc.

Untreated original biological samples are typically extremely complex by containing free carbohydrates, carbohydrates bound to proteins, lipids etc and also compounds which do not contain any carbohydrate structures. This will make the tagging reaction complex and also render it difficult to obtain useful mass spectral data for the carbohydrates of interest. Therefore untreated biological samples are often processed in order to obtain samples that are enriched with respect to the carbohydrate(s) of interest. Thus, if the only interest is in carbohydrates linked to a certain parent biomolecule or group of parent biomolecules, then the original samples are treated to obtain fractions or treated samples that are enriched in the biomolecules concerned. After release of the carbohydrates from the parent biomolecules, the so obtained free carbohydrates may be used as a starting sample in the present invention, if necessary, after further enrichment of the desired carbohydrates. By selecting the release conditions one can obtain free carbohydrates that have previously been attached to the parent biomolecule (for instance a protein) by an N- or an O-glycosidic bond.

In case the carbohydrates of interest are not conjugated to a parent biomolecule in the original biological sample, separation leading more or less directly to a fraction being enriched in the carbohydrate(s) of interest may be carried out. This kind of fractions may be used as starting samples.

If necessary the above-mentioned biologically derived samples are adjusted with respect to pH, salt concentration, carbohydrate concentration, etc before being used as samples provided in step (i.a).

The number (n) of carbohydrates to be quantified is ≧1, such as ≧2, ≧3, ≧4, ≧5, <6, ≧7, ≧8, ≧9, or ≧10. The upper limit may be 100, 200 or 300. The samples provided in step (i.a) might or may not contain other carbohydrates than those that are to be quantified. For samples comprising a large number of carbohydrates to be quantified there are typically included separation protocols which divide the starting sample into simpler samples which contains fewer carbohydrates to quantify. See under the heading “Separation” below.

The carbohydrates to be quantified preferably have a reactive group in common permitting tagging to take place. See below under the heading “Mass tagging reagent and tagging (Step (i.b))”. Suitable groups that can be utilised for tagging in carbohydrates are: hydroxy (primarily primary or secondary hydroxy), carboxy, caronyl (keto and aldehyde), amino etc.

Mass Tagging Reagents and Tagging (Step (b))

This step comprises reacting each of the samples provided in step (i.a) with a sample unique mass tagging reagent in order to introduce a mass tag (mass tag I, II, III etc) on each carbohydrate to be quantified. In other words carbohydrate A-I, carbohydrate B-I, carbohydrate C-I etc is formed in sample I; carbohydrate A-II, carbohydrate B-II, carbohydrate C-II etc is formed in sample II; etc.

In certain variants of the invention there may be used two or more different mass tagging reagents per sample for introducing different tags on carbohydrates having different kinds of reactive groups.

The mass tagged forms are formed “in a predetermined and reproducible manner” by which is meant that there is formed a predetermined amount of a tagged form of each of the carbohydrate(s) to be quantified in relation to the amount of the corresponding carbohydrate(s) in the starting sample provided in step (i.a). In preferred variants the mass tagging reagents and the conditions should be selected to give an essentially efficiency (constant yield) both with respect to the same reagent run at different occasions and for mass tagging reagents introducing different mass tags. In this context “constant yield” typically means 70%±30%, such as 70%±20% or 70%±10% based on the corresponding untagged carbohydrate. It may also be advantages to secure reproducibility by arranging so that the yield is quantitative, i.e. ≧80%, such as ≧90% or ≧95% based on the untagged carbohydrate. In preferred embodiments the tagging reagents have been selected so that the tagging reaction can be run under essentially equal conditions.

The differences in mass between the tags depend on (a) different elemental composition of the tags and/or (b) different isotope composition of one or more elements of the tags.

A mass tag (I, II, III etc) is defined as the complete group introduced on a carbohydrate by the mass tagging reagent/reaction.

The mass tags are often small compared to the molecular weight of the carbohydrate to be quantified. Most tags typically have a mass that is at most 50%, many times at most 10% such as at most 5%, of the mass of the heaviest of the carbohydrate(s) to be tagged. Mostly the optimal mass tags have molecular weights ≦1000 Dalton, such as ≦500 Dalton.

In both alternative (a) and (b) above, the difference in the masses of the tags introduced should be such that it results in distinct measurable peaks in the mass spectrum obtained in step (ii). Calculations in step (iii) will be facilitated and made more safe and reliable if the tags are selected such that base line separation of the peaks corresponding to the tagged forms of a carbohydrate is enabled. The optimal mass difference between the tags depends on various factors, for instance the mass spectrometer. The mass difference of two different tags of the same elemental composition is typically ≧2, such as ≧4 or ≧6 Dalton, for instance 7, 8, 9, 10, 11, 12 Dalton or ≧13 Dalton. An upper limit for this mass difference can be 25 Dalton. In the case the tags differ with respect to elemental composition the difference may be up to 200 Dalton or higher. If three or more tags are used the largest mass difference should be within the limits given above.

Typical isotopes that are useful in the present invention are: ¹H/²H/¹²C/¹³C, ¹⁴N/¹⁵N, ¹⁶O/¹⁷O/¹⁸O, ³²S/³⁴S, isotopes of Cl, Br and I and P etc.

The mass tagging reaction preferably takes place in a one-step reaction protocol, but may more typically involve a step-wise protocol including for instance a first activation step and a subsequent second step during which the groups causing the mass difference are introduced. Depending on the circumstances, also further steps, reactions and reagents may be needed. All the steps and reagents involved in tagging a carbohydrate are comprised within the terms “mass tagging reaction”, “tagging reaction”, “mass tagging reagent” and “tagging reagent” (except for the carbohydrate as such).In the case a carbohydrate does not contain any suitable reactive group permitting mass tagging such groups can be introduced in the mass tagging reaction.

Potential mass tagging reagents should have reactive groups matching reactive groups in the carbohydrates to be quantified.

For carbohydrates containing a carbonyl group, such as in an aldehyde or a ketone, the mass tagging reagents may have

(a) an (—NH—)—containing group that form adducts with carbonyl groups of aldehydes and/or ketones, or

(b) a reducing agent capable of reducing aldehydes and ketones to alcohols, while at the same time introducing a hydrogen isotope selected from hydrogen, deuterium or tritium.

The masses of the tags introduced in this manner differ as discussed above, with the proviso that variant (b) only gives mass tags that differ with respect to isotope composition.

The (—NH—)—containing group in variant (a) may be a primary or secondary amino group or a hydrazine derivative (e.g. acyl hydrazine, sulfonyl hydrazine etc).

In order to give a stable adduct, variant. (a) can be combined with a reducing agent converting the adduct to an amine. The reducing agent should preferably be selected to have a higher reducing activity towards the adduct than towards the free carbonyl group during the conditions applied. As a general guideline the selectivity of the reducing agent for reducing the adduct compared to reducing the aldehyde should be similar to or better than the selectivity expressed by NaBH₃CN.

Typical reducing agents to be used according to variant (b) includes sodium borohydrides, such as NaBH₄, NaBH₃CN and the like in which the hydrogen may be replaced with deuterium or tritium together with isotope variants of other hydrides that are capable of reducing aldehydes and ketones to alcohols.

For carbohydrates containing an amino group the mass tagging reagents may have an activated acid group, for instance an activated carboxy group. Potentially useful groups are reactive ester groups, acid halide groups, anhydride groups etc. The masses of the tags introduced in this manner differ as discussed above. The difference in mass for reagents of this kind is located to the “acid” part of the reagent, i.e. the part that becomes attached to the amino group.

For carbohydrates containing a carboxy group the mass tagging reagents typically have an (—NH—)—containing group, a hydroxy groups or any other reactive group that may be caused to react with an activated form of the carboxy group. The (—NH—)—containing group may be a primary or secondary amine, a substituted hydrazine (e.g. acyl hydrazine, sulfonyl hydrazine etc). Suitable hydroxy groups include alcoholic and phenolic hydroxy. The masses of the tags introduced in this manner differ as discussed above.

Other combinations of the reactive groups in the mass tagging reagents and in the carbohydrates to be quantified can be deduced from the chemical literature.

Catalytic reactions, for instance enzymatic reactions and enzymes for introducing the mass tag at a predetermined position in the carbohydrates to be quantified, are included in the concept of mass tagging reactions/reagents.

Quantification according to the invention will only be enabled for carbohydrates having the group permitting reaction with the mass tagging reagents used. In order to extend the quantification to carbohydrates not having this group but other reactive groups, a combination of different mass tagging reactions/reagents will have to be applied to the same sample. It is believed that the simplest way to accomplish this is to perform the sequence (i)-(iii) for each group on separate aliquots of the starting samples.

One way of securing predetermined reproducible amounts of tagged forms is to utilise an excess of the mass tagging reagent in relation to the sum of the total amount of carbohydrates to be quantified plus the amount of other constituents in the reaction mixture that consume mass tagging reagents. If using this principle, the typical excess could be at least 50%.

After the tagging reaction, it can be advantageous to remove unreacted tagging reagents. Depending on the circumstances this can be done at a position before step (ii) (mass spectrometry step), for instance on the individual reaction mixtures between step (i.b) and (i.c), on the combined sample between steps (i) and (ii). Removal can be accomplished by contacting the liquid containing an unreacted tagging reagent with a solid phase bound form of structures that are capable of interacting with unreacted tagging reagents. The solid phase may for instance be in the form of beads or other particles.

The tags should not contain structural elements that give the same or similar fragmentation pattern in MS as the carbohydrate to be quantified. This in particular applies if tandem MS is used in step (ii). Thus the tag should not exhibit carbohydrate structure of the same kind as those present in the carbohydrates to be quantified.

Combining tagged forms derived from different starting samples (step i.c)

This step typically comprises mixing defined aliquots of the reaction mixtures obtained in step (i.b). Preferably the aliquots are equal for each sample. This does not exclude that the various tagged forms produced in step (i.b) are enriched or excess reagents removed before tagged forms are combined to form the combined sample.

Steps (ii)-(iii). Mass Spectrometry and Quantification

These steps comprise subjecting the mixtures of mass tagged forms of each carbohydrate to be quantified to mass spectrometry. From the relation between the signals (peaks) for the mass tagged forms which are present in a mixture corresponding to a certain carbohydrate, the amount of each mass tagged form relative to any other of the mass tagged forms in the same mixture can be determined. The relative amounts for the mass tagged forms in the mixture can then give the relative amounts of the corresponding untagged carbohydrate in the starting samples. The principles for performing these calculations have been previously practised in the field with respect to the quantification of proteins. See Aebersold et. al (WO 0011208); Gygi et al (Nature Biotechnology 17 (1999) 994-999); Münchbach et al (Anal. Chem. 2000 (72) 4047-4057) and Oda et al (Proc. Natl. Acad. Sci. USA 96 (1999) 6591-6596).

In the case there are used a control sample or a reference sample which contains known absolute amount of carbohydrates, the method will be able to give the absolute amount(s) or absolute concentration(s) of the carbohydrate(s) to be quantified

The term “mass spectrum” in this context refers both to the set of signals for the mass tagged forms obtained from the mass spectrometer and to the representation of these signals as peaks in a conventional mass spectrum.

Typically the mass tagging reaction will introduce only one tag on each carbohydrate to be quantified. In some cases when the reactive group occurs more than once in a carbohydrate there may be introduced two, three or more identical tags on each carbohydrate. The signals (peaks) to be used in a mass spectrum for quantification in accordance with the invention will in the normal case be positioned at mass differences that are the same as the mass differences of the tags. If more than two identical mass tags are introduced per carbohydrate also signals that are located at mass differences that are twice, three times etc the mass differences of the tags used can be utilised.

In the case the salt concentration in the combined sample to be used in step (ii) is too high for the mass spectrometry step, there may be included one or more desalting steps before step (ii). As an alternative one or more of the preceding steps may be precautionary adapted to conditions giving a sufficiently low salt concentration in the sample for a high qualitative mass spectrometry step (ii).

For ionisation the mass-spectrometry may utilise electrospray (ESI), matrix associated laser induced dissociation (MALDI) etc of the tagged forms in the individual fractions. Tandem MS (MSⁿ) may be used in case there is a need to sequence the tagged carbohydrate fragments that are subjected to the mass spectrometry step. Depending on the need the mass spectrometry step set up maybe in form of LC-MS, CE-MS etc. See also under the heading “Separation steps” below.

The basis for the quantification in step (iii) is that:

(a) the signal in a mass spectrum is a function of the amount of the tagged form applied in the mass spectrometer, and that

(b) this amount in turn is a function of the amount of the untagged carbohydrate in the starting sample from which the tagged form derives.

When calculating the amounts in step (iii) one has to take into account, for instance,

(1) the relative volumes of the starting samples provided in step (i.a),

(2) the relative dilution caused by forming the different reaction mixtures in step (i.b),

(3) relative tagging efficiency of the various tagging reagents in step (i.b),

(4) relative volumes and/or relative amounts of mixtures combined in step (i.c).

In a preferred case at least one of the following features applies:

(i) all the starting samples in step (i.a) have essentially equal volumes and/or essentially the same total concentration of carbohydrates to be quantified,

(ii) the dilutions and the tagging efficiency in the different reaction mixtures in step (i.b) are essentially equal, and

(iii) the volumes of the reaction mixtures and/or the amounts of the mixtures combined in step (i.c) are essentially equal.

Other Steps

There may be one or more additional steps before step (ii), for instance between step (i.a) and step (i.b). Illustrative examples are derivatisation steps for other purposes than mass tagging, separation steps and digestion steps etc.

Separation Step

The starting samples provided in step (i.a) might contain complex mixtures of carbohydrates that are difficult to quantify by the use of mass spectrometry in steps (ii)-(iii). The complexity may reside in the presence of a large number of different carbohydrates and/or carbohydrates having very small differences in masses and/or carbohydrates giving rise to similar fragments in a mass spectrometer etc. In these cases there may be inserted a separation step after step (i.b) but before step (ii) which enables coseparation of tagged forms of individual carbohydrates or groups of carbohydrates into fractions in which the quantification in step (iii) is simplified.

in case a separation step is included after step (i.b), the separation protocol applied and the tags introduced should be adapted to each other in such a way that tagged forms of the same carbohydrate coseparates. In other words to arrange so that tagged forms of the same carbohydrate is collected in the same fraction.

The separation step may encompass two or more separation protocols. In order to obtain a sufficiently high resolution it is advantageous that at least two of these separation protocols differ with respect to the separation principles employed.

Typical separation principles are separations based on differential interactions between the carbohydrates and a surrounding medium. The differences may, for instance, be reflected by differences in size and/or charges of the carbohydrates, i.e. differences that are utilised in size exclusion chromatography, adsorption chromatography, gel electrophoresis e.g. PAGE, capillary electrophoresis, isoelectric focusing e.g. in gels, capillaries, etc. The differences may also be reflected in a differential affinity of the carbohydrates with one or more ligands attached to the separation media used etc. Differential affinity may be utilised in either or both of the adsorption step and the desorption step.

The concept of different principles is also defined by the way in which the mass transport is talking place during the separation. Illustrative examples by liquid flow such as in chromatographic procedures, by an applied electric field such as in electrophoresis, by centripetal force such as in centrifugation, are by stirring or by other means giving turbulence/agitation such as in batch-designed procedures, by gravity etc.

Separation protocols that are based on the same separation principles include that a separation protocol is run on a fraction obtained in a previous protocol under essentially the same conditions as in the previous protocol. It also includes variations between different protocols, such as changes in pH, concentration of salts and the like etc.

As a general rule a more complex mixture will require a total higher resolving capacity on the combination of protocols selected. Preferred protocols, either alone or in combination, are reversed phase liquid chromatography, separation according to isoelectric points (isoelectric focusing etc), and molecular size (gel electrophoresis etc). The separation step thus may be multidimensional, i.e. contains two or more separation protocol utilising different principles, such as in 2-D gel electrophoresis (isoelectric focusing in the first dimension and gel electrophoresis in the second dimension).

A possible fractionation step inserted before step (ii) may also include one or more of the various fractionation steps mentioned under the heading “Fractionation steps” below.

In many commercially available mass spectrometers a separation step is integrated, for instance LC-MS, CE-MS etc. LC stands for liquid chromatography typically reverse phase liquid chromatography. CE stands for capillary electrophoresis.

Other Derivatisation Steps

At certain positions in the sequence (i.a.)-(i.c) there may also be inserted derivatisation steps for the purpose of

(1) enhancing the ionisation during the mass spectrometry step (ii) (at any position preceding step (ii)),

(2) enhancing the fragmentation during the mass spectrometry step (ii) (at any position preceding step (ii),

(3) minimising separation differences introduced by the mass tag (at any position preceding a separation step), and

(4) introducing an affinity handle to be used for selectively fishing out carbohydrates that are derivatized,

(5) introducing an analytically detectable label for instance a radiation emitting group, for instance a fluorophore or a groups that is possible to detect by UV.

Items (4) and (5) are primarily intended for facilitating and monitoring separation steps that may be included according to the invention.

Other kinds of derivatisations are predictable, for instance utilising enzymes for modifying specific saccharide units in order to enable or improve mass spectrometry (MS) detection and quantification of such units.

For each of these additional derivatisations the same reagent is preferably used for all the samples. A potentially important variant is to design the mass tagging reagent so that one or more of these derivatisations are accomplished when the mass tagging reaction is carried out See for instance the experimental part where the mass tags introduced combine a mass tagging function with an ability to work as an affinity handle and/or with good fluorescent or UV properties.

Digestion Step

This kind of steps may be utilised as outlined in SE patent application 0003566-7, filed Oct. 2, 2000 which is hereby incorporated by reference.

A digestion steps may be performed enzymatically or by chemical means. It may be inserted between step (i.a.) and step (ii) with modifications of the subsequent steps as outlined in SE patent application 0003566-7, filed Oct. 2, 2000.

The Second Aspect of the Invention

The second aspect of the invention is a kit for the analysis of compounds exhibiting carbohydrate structures in which there is a free carbonyl group in form of an aldehyde or a ketone group. The preferred compounds to be analysed by the use of the kit are free carbohydrates, i.e. carbohydrate compounds not covalently attached to a peptide or lipid structure and preferably have oligosaccharide structure. The kit is characterised in that it comprises

(a) two, three or more mass tagging reagents each of which are capable of forming an adduct with a carbonyl group selected from aldehyde and ketone groups, said adduct comprising a mass tagging group that derives from one of the mass tagging reagents and has a mass that is different for the different adducts formed by use of said two, three or more mass tagging reagents, and

(b) optionally a reducing agent for stabilising the adducts formed by the use of said two, three or more mass tagging reagents.

The mass tagging reagents and the reducing reagent are selected according to the same principles as given above under the heading “Mass tagging reagent and tagging (Step (i.b)”.

The mass tagging reagents and the mass tags may comprise a member (ligand) of an affinity pair which preferably is the same for all the mass tagging reagents and mass tags. In this variant of the second aspect the kit may also comprise an affinity reagent which is an affinity counterpart to said member (receptor).

A ligand-receptor pair is a pair of reactants that are capable of binding to each other via a so-called affinity reaction. This kind of reactions is typically illustrated by so called biospecific affinity reactions or similar reactions in which synthetically produced affinity pairs are binding to each other via affinity. Affinity pairs (ligand-receptor pairs) are illustrated by antigen/hapten and antibodies, biotin and streptavidin, IgG and IgG-binding proteins that bind to the Fc part of IgG, lectins and compounds exhibiting carbohydrate structures etc. One member (a ligand) of an affinity pair can be part of the mass tag while the other member (the receptor) of the pair then is part of the affinity reagent of the kit. The term antibody includes antigen/hapten-binding fragments and modifications of antibodies (Fab-fragments, single chain antibodies etc).

In the experimental part, mass tags that can function as affinity handles are illustrated by biotin and hapten molecules. In particular hapten molecules may comprise an aromatic ring system that is substituted with electron donating and/or electron-accepting groups containing a free electron pair and/or π-electrons that are able to delocalise by resonance to the aromatic system.

The kind of structures defined in the preceding paragraph is often chromophores. They can be used for making qualitative and quantitative determinations of individual carbohydrates after labelling and separation of the carbohydrates as discussed above. For this kind of use there is no need for mass spectrometry. In case they are fluorescent for instance with a molar extinction coefficient >1000 cm⁻¹M⁻¹ such as >10000 cm⁻¹M⁻¹ the determinations may be carried out by fluorometry.

In a sub-aspect of the invention at least one, two, three or more of the tagging reagents of the kits are bifunctional in the sense that they contain both a chromophore structure and an a separate affinity handle, both of which may be as defined above. This kind of reagents has been described by Shinohara in (Anal. Chem. 68 (15) (1996) 2573-2579). The invention will know be illustrated in the experimental part that also provides proof of the principle underlying the invention. The invention is defined in the appending claims.

### DETAILED DESCRIPTION OF THE DRAWINGS

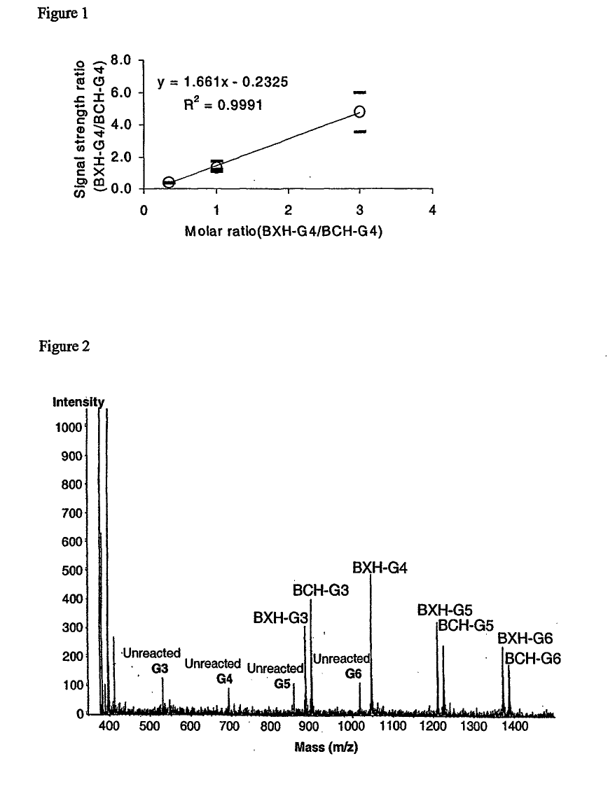

FIG. 1 shows the correlation between molar ratio (bxh-g4/bch-g4) and signal strength ratio (n=2 or 3). Open circles correspond to mean value while bars indicate individual data. A trend line was drawn for the mean values. The equation and the r-squared value is given.

FIG. 2 shows an MS-spectrum obtained showing differential display of oligosaccharides using two tagging reagents: BCH and BXH.

### Experimental Part

The present examples are given only to illustrate the invention and are not intended to limit its scope as defined by the appended claims.

1. Tagging of Oligosaccharides

(a) Tagging by Borohydride (NaBH₄) and Sodium Borodeuteride (NaBD₄)

Oligosaccharides are dissolved in 200 μl of 0.05N NaOH, and reduced by incubation with NaBH₄ or NaBD₄ (SM excess, dissolved in dimethylformaide) at 37° C. for 4.5 hr. The reaction is stopped by adding 100 μl of 1M acetic acid and evaporated to dryness. The residue is evaporated with water five times.

(b) Tagging by ethyl p-aminobenzoate (ABEE) and n-butyl p-aminobenzoate (ABBE)

Solutions of ABEE or ABBE solution (2 μl, 1M, in AcOH/DMSO (3:7)) was added to 0.5 μl of an aqueous solution of containing ca. 1 nmol of oligosaccharides. The reaction tubes were then vortexed and maintained at 90° C. for 30 min. Sodium cyanoborohydride (1.8 μmol) in 5 μl of water was added to the reaction mixture, and the reaction mixture was maintained at this temperature for an additional hour.

(c) Tagging by N⁶-biotinoyl-2,6-diaminohexanoic Acid Hydrazine (BCH), N⁶-biotinoyl-6-aminohexanoic Acid Hydrazine (BXH), Dansyl Hydrazine, 7-diethylaminocoumarin-3-carboxylic Acid Hydrazide (DCCH) and N-(9-fluorenylnethoxycarbonyl) Hydrazine (FMOC Hydrazine)

Oligosaccharides are dissolved in 10 μl of water, and mixed with the hydrazine derivatives dissolved in water or acetonitrile (1-4 μM excess) and incubated at 90° C. for 1.5 hr. Cooling the reaction tube in an ice bath stops the reaction.

2. Differential Display by MS Using Structurally Similar Tagging Reagents

Mixtures of maltooligosaccharides (G3, G4, G5 and G6) mixtures were tagged with BCH and BXH. These tagging reagents are structurally similar and the difference in molecular weight is ca. 15 Da. For G5 and G6 the tagging yields for BCH and BXH were >85% and >92%, respectively.

In order to compare the ionic strengths and ion species of BCH-tagged and BXH-tagged oligosaccharides in the MALDI/TOF analysis, maltotetraose (G4) tagged with BCH or BXH according to (c) above were mixed in three different proportions (BCH-G4/BXH-G4=1:1, 1:3 and 3:1). In the MALDI-TOF analysis using DHB (2,5-dihydroxy benzoic acid) as matrix, both BCH-G4 and BXH-G4 produced only MNa⁺ species (Maldi-TOF instrument from Amersham Pharmacia Biotech AB, Uppsala, Sweden). Although the BXH-tagged form tends to give slightly higher signal strength than the BCH-tagged form for equimolar amounts, the two tagged forms were considered comparable on an equimolar basis. See FIG. 1.

A mixture of equimolar amounts (50 nmol) of each of G3, G4, G5 and G6 was tagged with BXH, and a mixture of 100n mol of G3 and 50 nmol of each of G5 and G6 was tagged with BCH. After the tagging, both reaction mixtures were mixed and provided for MALDI-TOF analysis. As shown in FIG. 2, all tagged forms of G3, G5 and G6 could be observed as doublet signals with a m/z difference of 15, while G4 could be observed as a single signal. The relative signal strength was higher for BCH-G3 than for BXH-G3, while the opposite trend was observed for tagged forms of both G5 and G6. This observation agrees with the true relative quantities existing in the mixtures.

1. A method for quantification of one or more carbohydrates in two or more starting samples, characterised in that it comprises the steps of:
(i) providing a combined sample containing for each carbohydrate a mixture of one or more mass tagged forms derived from the carbohydrate, wherein each of said one or more mass tagged forms in the mixture
comprises a mass tag that is unique for the starting sample from which its carbohydrate part is derived, and
is present in the combined sample in an amount that relates to the amount of the carbohydrate in the starting sample from which its carbohydrate part is derived;
(ii) subjecting each mixture of mass tagged forms to mass spectrometry to obtain a mass spectrum;
(iii) quantifying from signals of mass tagged forms in the mass spectrum the amount of a carbohydrate to be quantified in one original sample relative to the amount(s) of the same carbohydrate in one or more of the other original samples.

2. A method according to claim 1, characterised in that step (i) comprises
(a) providing said two or more starting samples;
(b) treating each of the starting samples with a sample unique mass tagging reagent which is capable of transforming each of said carbohydrates to a mass tagged form of the carbohydrate;
(c) combining the mass tagged forms obtained in step (b) to a combined sample containing the different mixtures of mass tagged forms provided in step (i).

3. A method according to claim 1 or 2, characterised in that at least one of said original samples is a reference or control sample, for instance containing a predetermined amount of one or more of said carbohydrates.

4. A method according to claim 1 or 2, characterised in that the mass tags differ in elemental composition and/or isotope composition.

5. A method according to any of claims 1-4, characterised in that
(A) there is a separation step between step (i.b) and step (ii) resulting in one or more fractions, each of which is enriched with respect to mass tagged forms of at least one of the carbohydrates to be quantified in relation to mass tagged forms of other carbohydrates to be quantified, and
(B) step (ii) is carried out on a combined sample derived from a fraction obtained according to (A).

6. A kit of reagents, characterised in that it comprises:
(a) two, three or more mass tagging reagents each of which are capable of forming an adduct with a carbonyl group selected from aldehyde and ketone groups, said adduct comprising a mass tagging group that derives from one of the mass tagging reagents and has a mass that is different for the different adducts formed by use of said two, three or more mass tagging reagents;
(b) optionally a common reducing agent for stabilising the adducts formed by the use of said two, three or more mass tagging reagents.

7. A kit according to claim 6, characterised in that each of the mass tagging reagents and the mass tags comprise a member of an affinity pair which is the same for all the mass tagging reagents and mass tags, and that the kit also comprises an affinity reagent which is an affinity counterpart to said member.

8. A kit according to claim 6 or 7, characterised in that said mass tagging reagents and mass tags comprise biotin.

9. A kit according to claim 6 or 7, characterised in that said mass tagging reagents and mass tags comprise an aromatic ring systems that is substituted with electron donating and/or electron-accepting groups containing a free electron pair and/or π-electrons that are able to delocalise by resonance to the aromatic system.
